(19) United States
(12) Patent Application Publication
(10) Pub. No.: US 2026/0256487 A1
(43) Pub. Date: Sep. 3, 2026

(54) TONGUE CLEANER

(71) Applicant: Seokjin LEE, Yangju-si (KR)

(72) Inventor: Seokjin LEE, Yangju-si (KR)

(21) Appl. No.: 18/872,912

(22) PCT Filed: Sep. 21, 2022
(86) PCT No.: PCT/KR2022/014084

§ 371 Date: Dec. 9, 2024

(30) Foreign Application Priority Data
Jun. 14, 2022 (KR) 10-2022-0072189
Aug. 30, 2022 (KR) 10-2022-0109276

(51) Int. Cl. A61B 17/24; A61B 17/00

(52) U.S. Cl. CPC A61B 17/244; A61B 2017/0042

(57) ABSTRACT

The present invention relates to a tongue cleaner, and more specifically, to a tongue cleaner which, in addition to removing tongue plaque, provides tongue massaging and cleaning functions simultaneously and in a complex manner through a head, thereby enabling hygiene and health management for the tongue, and in particular, the head is configured to be attachable to and detachable from a body having a simplified frame structure so as to make attachment and detachment of the head easier and to enable the tongue cleaner to be more cleanly managed, and the simplified structure enables significant reduction in production cost, and thus, through the low cost, expansion of supply and high efficiency can be expected.

### TECHNICAL FIELD

The present disclosure relates to a tongue cleaner, and more specifically, to a tongue cleaner which, in addition to removing tongue plaque, provides tongue massaging and cleaning functions simultaneously and in a complex manner through a head, thereby enabling hygiene and health management for the tongue, and in particular, the head is configured to be attachable to and detachable from a body having a simplified frame structure so as to make attachment and detachment of the head easier and to enable the tongue cleaner to be more cleanly managed, and the simplified structure enables significant reduction in production cost, and thus, through the low cost, expansion of supply and high efficiency can be expected.

### BACKGROUND

For effective oral care, it is preferable to use dental floss, interdental brushes, gargle, gum massage, and whitening in addition to brushing, and in particular, it is important to keep the tongue clean by using a tool to remove the tongue plaque (hereinafter, referred to as a tongue cleaner), in order to clean the tongue plaque that builds up on the tongue.

In particular, unlike teeth, the tongue is made of soft tissue, and its surface is rough overall with mucosa protruding.

This area is called a lingual papilla, and the lingual papillae are distributed in greater numbers towards the root (inner) part of the tongue, and the surface cells of the lingual papillae distributed on the tongue gradually keratinize and fall off, which acts as a major factor in forming tongue plaque. In particular, the area corresponding to the inner ⅓ of the tongue (root) has a rougher surface than the tip of the tongue, making it the area where tongue plaque composed of saliva, food, oral microorganisms, and epithelial cells that have fallen within the oral cavity, including the lingual papilla, easily accumulates. Once tongue plaque has accumulated in this way, it is difficult to remove the tongue plaque within the oral cavity without a physical cleaning process.

Accordingly, in order to effectively manage oral hygiene, it is very important to remove the tongue plaque. However, since a toothbrush, which is designed to brush teeth and gums, cannot properly remove the tongue plaque in this area, it is recommended to use a specialized tongue cleaner that is suitable for the three-dimensional shape and tissue elasticity of the tongue and takes into account the characteristics of the lingual papilla.

Therefore, conventional technologies (e.g., Korean Laid-open Patent Publication No. 10-2022-0033789, Korean Patent No. 10-2049109) have been proposed in which oral care accessories with various functions, such as the tongue cleaner, the interdental brush, and the dental floss, are integrated into appropriate locations on a general toothbrush body, or each accessory is configured to be detachable (hereinafter referred to as a replaceable structure).

However, in the case of the replaceable structure, it is common to have a rather complex structure to more strongly implement the detachable connection between the main body and the accessory in order to prevent the accessory from being arbitrarily disassembled in the oral cavity during the process of use. Accordingly, not only did it not ensure convenience of use because the accessory was not easy to attach and detach, but there was also the problem that hygiene care was somewhat difficult due to the complex structure.

In addition, due to its complex structure, the production cost increases, and as a result, in terms of the oral care product being a consumable product, it is reality that the utility value of consumable oral care products is not high in terms of cost.

### PRIOR ART DOCUMENTS

### Patent Documents

Korean Laid-open Patent Publication No. 10-2022-0033789

Korean Patent No. 10-2049109

### SUMMARY

### Technical subject

The present disclosure is to solve the above-mentioned problems of the prior art, and provides a tongue cleaner, and more specifically, to a tongue cleaner which, in addition to removing tongue plaque, provides tongue massaging and cleaning functions simultaneously and in a complex manner through a head, thereby enabling hygiene and health care for the tongue, and in particular, the head is configured to be attachable to and detachable from a body having a simplified frame structure so as to make attachment and detachment of the head easier and to enable the tongue cleaner to be more cleanly managed, and the simplified structure enables significant reduction in production cost, and thus, through the low cost, expansion of supply and high efficiency can be expected.

### Problem to be Solved

The present disclosure provides a means for achieving the above object, comprising: a hard body formed in a frame shape with a mounting space formed penetrating therein; and a soft head detachably coupled to the mounting space to clean and massage the tongue; wherein the body includes: a handle frame formed with a first mounting space formed therein; and a head frame formed extending from an upper end of the handle frame and forming a second mounting space formed inside to communicate with the first mounting space; wherein the body formed the frame shape is formed of an elastically deformable material such that, upon compressive deformation of the handle frame, the head frame is compressively deformed in conjunction therewith, and thus, the head coupled to the second mounting space is compressed and fixed within the second mounting space.

According to another preferred embodiment of the present disclosure, a scraper protrusion for removing tongue plaque may be formed on the upper end of the main body.

According to another preferred embodiment of the present disclosure, at least one head may be coupled and stored in a single row in the first mounting space.

According to another preferred embodiment of the present disclosure, a support may be formed at the bottom of the first mounting space.

### Effect of Invention

The present disclosure provides an advantageous effect regarding the tongue cleaner which, in addition to removing the tongue plaque, provides the tongue massaging and cleaning functions simultaneously and in the complex manner through the head, thereby enabling the hygiene and health care for the tongue, and in particular, the head is configured to be attachable to and detachable from the body having the simplified frame structure so as to make the attachment and detachment of the head easier and to enable the tongue cleaner to be more cleanly managed, and the simplified structure enables the significant reduction in the production cost, and thus, through the low cost, expansion of supply and high efficiency can be expected.

### BRIEF DESCRIPTION OF THE DRAWINGS

FIG. 1 shows the overall appearance of the tongue cleaner according to the present disclosure.

FIG. 2 shows the disassembled configuration of the tongue cleaner according to the present disclosure.

FIG. 3 is an exemplary drawing of the configuration regarding a rear surface of the main body.

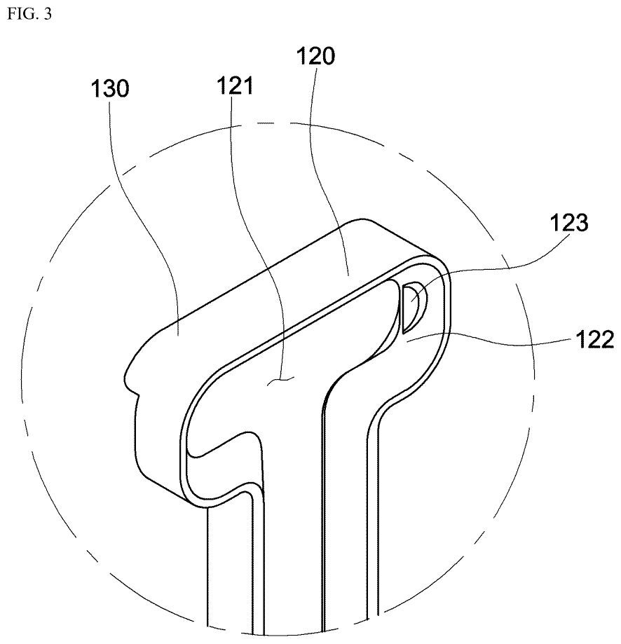

FIG. 4 shows the front configuration of the main body.

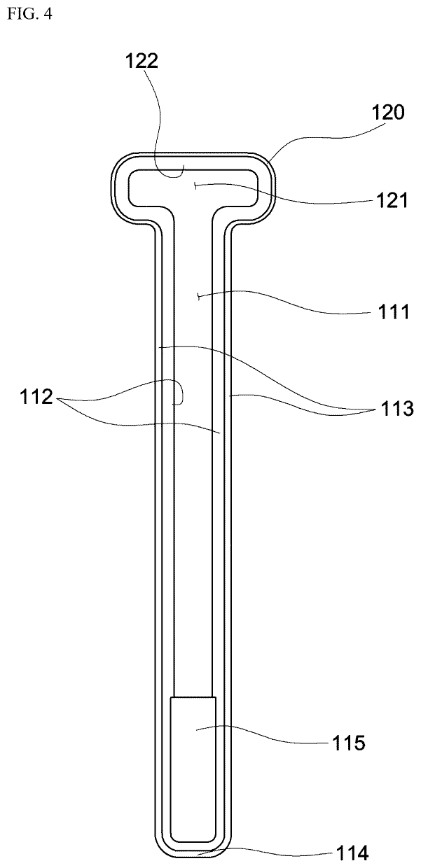

FIG. 5 shows the operation of the tongue cleaner according to the present disclosure.

FIG. 6 shows a partial cut state of the main body.

FIG. 7 shows the planar configuration of the head.

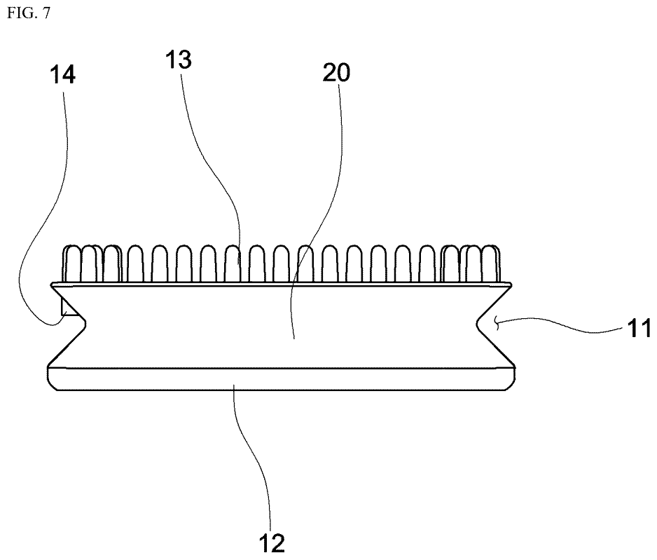

FIG. 8a shows a cross-sectional configuration based on the line A-A illustrated in FIG. 1, and FIG. 8b shows a cross-sectional configuration based on the line B-B illustrated in FIG. 1.

FIG. 9 illustrates the configuration regarding the rear surface according to various embodiments of the head.

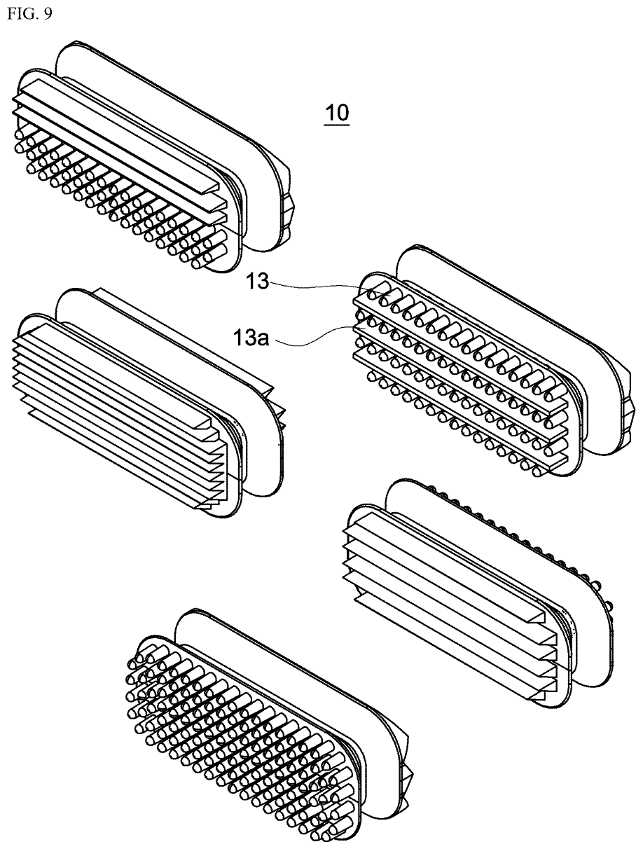

FIG. 10 shows one side of the upper structure of the tongue cleaner according to the present disclosure.

FIG. 11 shows the storage state of the tongue cleaner.

FIG. 12 shows the state of use of the tongue cleaner.

### THE BEST FORM FOR THE IMPLEMENTATION OF THE INVENTION

In order to fully understand the present disclosure, preferred embodiments of the present disclosure will be described with reference to the accompanying drawings. The embodiments of the present disclosure may be modified in various forms, and the scope of the present disclosure should not be construed as being limited to the embodiments described in detail below. The embodiments are provided to more fully describe the present disclosure to those skilled in the art. Therefore, the shapes of elements in the drawing may be exaggerated to emphasize a clearer explanation. It should be noted that in each drawing, the same element may be illustrated as the same reference numerals. Detailed descriptions of known functions and configurations that may unnecessarily obscure the gist of the present disclosure are omitted.

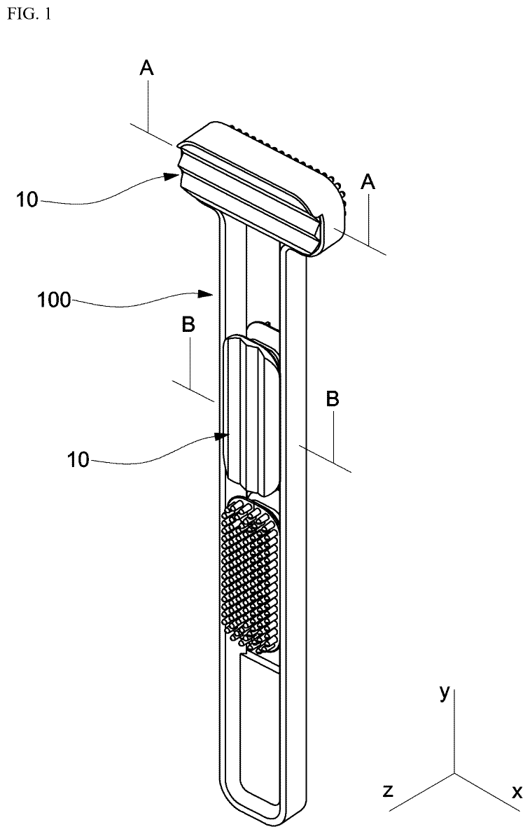

Prior to the description of the invention, for convenience, if a non-strict directional reference is specified with reference to the accompanying drawings, in the state as shown in FIG. 1, up and down (y), left and right (x), and front and rear (z) are divided, and in the description related to other drawings, the direction is specified and explained according to this reference.

The tongue cleaner according to the present disclosure has a structure that enables more efficient oral hygiene care by performing not only the functions of a general tongue cleaner, such as removing the tongue plaque, but also cleaning, massaging, and delicate finishing of the tongue all at once, while ensuring the convenience of oral hygiene care.

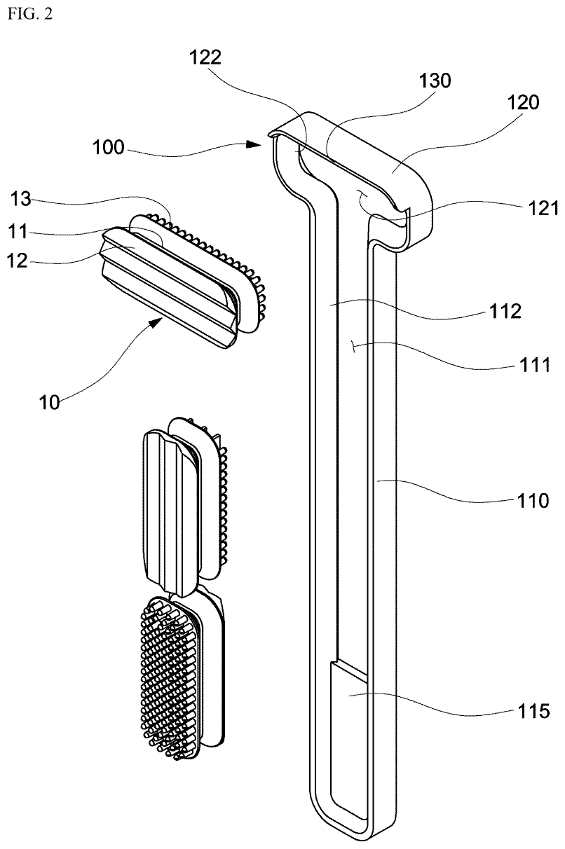

To this end, as shown in FIGS. 1 and 2, the configuration of the tongue cleaner according to the present disclosure can be broadly defined as including the main body (100) and the head (10).

The main body (100) is configured to act as a handle for a user to grip with their hands and can detachably support various heads (10).

The scraper protrusion (130) for removing the tongue plaque may be formed on the upper part of the main body (100), and the head (10) may be detachably coupled to the lower side of the scraper protrusion (130) so that the scraper protrusion (130) and the head (10) may come into contact with the surface of the tongue at the same time.

When the tongue cleaner is swept downward, the scraper protrusion (130) and the head (10) come into simultaneous contact with the tongue, the head (10) acts on the surface of the tongue, and the scraper protrusion (130) makes contact with the area through which the head (10) passed.

At this time, it is preferable that the scraper protrusion (130) and the head (10) be formed with different hardnesses. Normally, in order to remove the tongue plaque, it is effective to physically stimulate the surface of the tongue. Therefore, the scraper protrusion (130) may be formed with a linear protrusion structure having appropriate strength and hardness depending on the material of the main body (100) which is integrally molded with a hard synthetic resin material. In addition, the head (10) may be formed with a soft material, such as silicone or rubber, having a lower strength and hardness than the scraper protrusion (130) in consideration of the three-dimensional shape of the tongue, elastic tissue characteristics, etc., and in order to more effectively perform the cleaning and gentle massage actions.

More preferably, the head (10) is made of heat-resistant silicone to enable hot water disinfection.

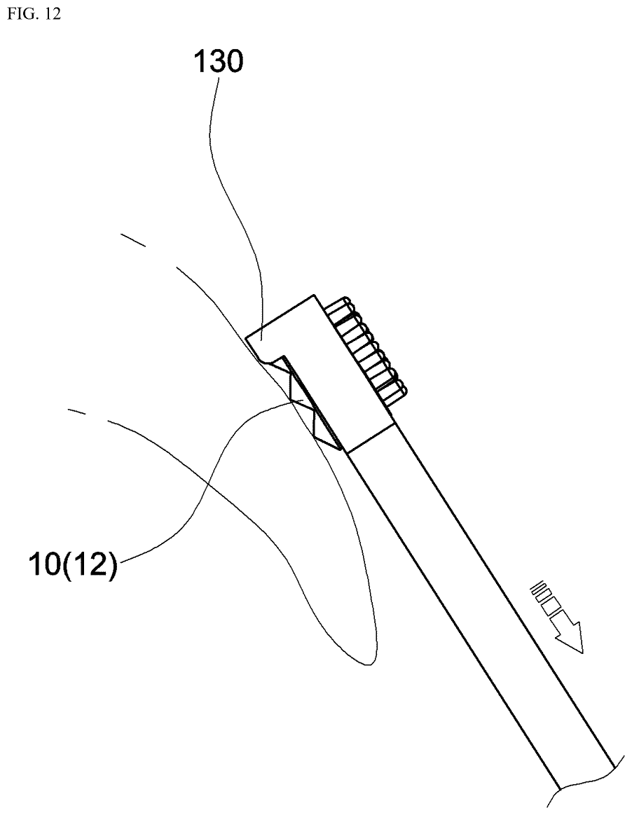

Accordingly, as illustrated in FIG. 12, when the tongue cleaner is used to sweep from the inside of the tongue to the tip of the tongue, the head (10) performs a primary cleaning and massaging action, and then the scraper protrusion (130) sweeps down more strongly along the position that the head (10) passed, thereby gradually removing the tongue plaque that was not removed by the head (10). Accordingly, not only is the tongue plaque removal and cleaning efficiency improved, but also the physical stress applied to the tongue can be minimized by sufficiently activating the tongue surface with a weak stimulus through the head (10) before the scraper protrusion (130) applies a strong stimulus to the tongue, thereby exposing it to a stronger stimulus through the scraper protrusion (130).

More specifically, the main body (100) may be formed in a frame shape so that the mounting space can be formed penetrating the interior, and a head (10) may be detachably coupled to the mounting space.

Accordingly, both the front and the rear of the head (10) combined inside the main body (100) having the frame shape can be formed to be exposed to the outside, and both the front and the rear of the exposed head (10) can be alternately used for tongue hygiene care.

The main body (100) may be exemplified as including, for example, the handle frame (110) in which the first mounting space (111) is formed, and the head frame (120) extending from the top of the handle frame (110) and having the second mounting space (121) formed within it, communicating with the first mounting space (111).

At this time, the main body (100) has a roughly ‘T’-shaped ergonomic structure, for example, the handle frame (110) is elongated up and down so that the user can easily grip and manipulate it with his or her hand, and the head frame (120) is elongated from the top of the handle frame (110) to the left and right so that it has a structure corresponding to the width of the tongue and can form a wider contact area with the tongue.

Here, a ‘T’-shape does not necessarily mean that the shape of the head frame (12) is in a straight line, but also includes shapes such as a semicircle, circle, oval, and polygon.

In addition, the first mounting space (111) and the second mounting space (121) may also be implemented as a ‘T’-shaped space as a single channel connected to each other.

Meanwhile, the head (10) may be detachably coupled to each of the first mounting space (111) and the second mounting space (121). The head (10) coupled to the first mounting space (111) is for storage or replacement, and the head (10) coupled to the second mounting space (121) is for acting directly on the tongue.

Alternatively, if the user uses only the scraper protrusion (130) formed on the top of the main body (100) with the head (10) separated, the first mounting space (111) can be used to store the separated head (10).

In other words, the first mounting space (111) acts as a storage box. In this case, at least one head (10) may be coupled and stored in a row along the longitudinal direction in the first mounting space (111) and combined for storage, and only a single head (10) may be combined in the second mounting space (121).

A plurality of heads (10) are configured to have different strength and hardness characteristics, respectively, and are distinguished by color, etc., so that the appropriate head (10) can be visually distinguished and easily replaced according to the situation.

In other words, the user can appropriately replace the head (10) of the second mounting space (121) with the head (10) coupled to the first mounting space (111) according to the situation, and in this case, in order for the head (10) to be properly coupled to the main body (100), fixing protrusions (112, 122) are formed to protrude along the circumferential direction on the inner surface of the main body (100), and the edge of the head (10) has a structure in which a fixing groove (11) corresponding to the fixing protrusions (112, 122) is recessedly formed.

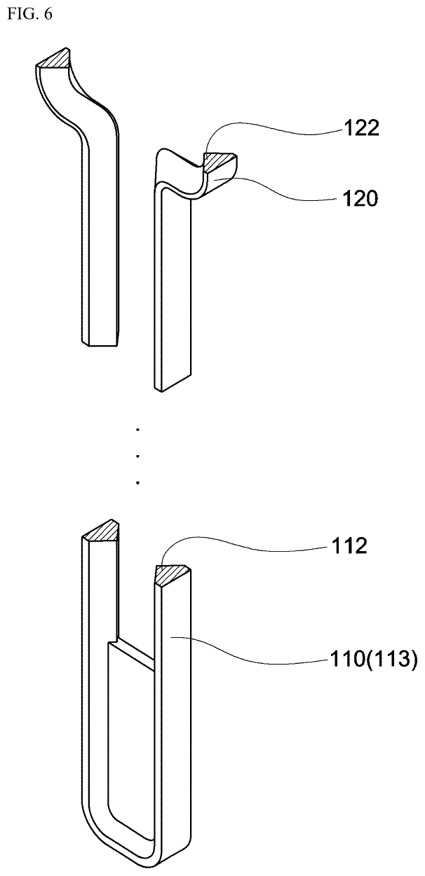

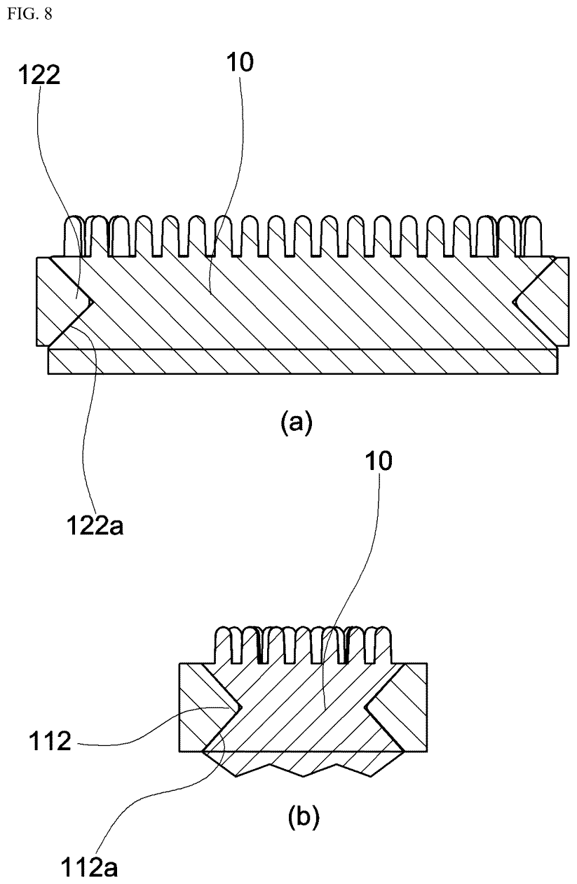

As shown in FIGS. 6 and 8, the fixing protrusions (112, 122) are formed in a triangular shape with their vertices oriented toward the center of the mounting space, so that the head (10) can be guided by the inclined surfaces (112a, 122a) formed in front and behind the fixing protrusions (112, 122) during the process of attaching and detaching the head (10).

In other words, when the head (10) is coupled to the main body (100), as line contact is made on the inclined surfaces (112a, 122a), the head (10) is guided to the center of the mounting space, and when the head (10) is forcibly fitted inward in this state, the head (10) is elastically deformed to fit the volume of the mounting space and enters the mounting space, and when it is completely seated in the mounting space, it returns to its original state, and the fixing protrusions (112, 122) and the fixing groove (11) are interlocked with each other in a groove-protrusion structure, so that the head (10) does not easily fall out in the front and rear directions.

In particular, the head (10) may be fixedly coupled in the second mounting space (121) in the front and rear direction as well as in the up and down and left and right directions, and in the first mounting space (111), it may be coupled so as to be able to slide up and down along the longitudinal direction of the first mounting space (111), so that the order or position of the head (10) for storage can be appropriately varied to suit preference.

Furthermore, the head (10) coupled to the second mounting space (121) also has the advantage that the head can be more hygienically maintained, since both the front and the rear are exposed to the outside.

As explaining the main body (100) more specifically, it is explained that the handle frame (110) may consist of, for example, a pair of vertical frames (113) that constitute the first mounting space (111), spaced apart from each other at a certain distance on the left and right. In this case, the fixing protrusion (112) may be formed on the surface where each of the vertical frames (113) faces each other, and the lower ends of the pair of vertical frames (113) may be connected to each other through a connecting frame (114).

In the case of the head frame (120), the structure is not particularly limited, but according to the embodiment of the attached drawing, it is bent multiple times in a pair of vertical frames (113) to form a rectangular structure so that the main body (100) may have a frame shape connected as one frame.

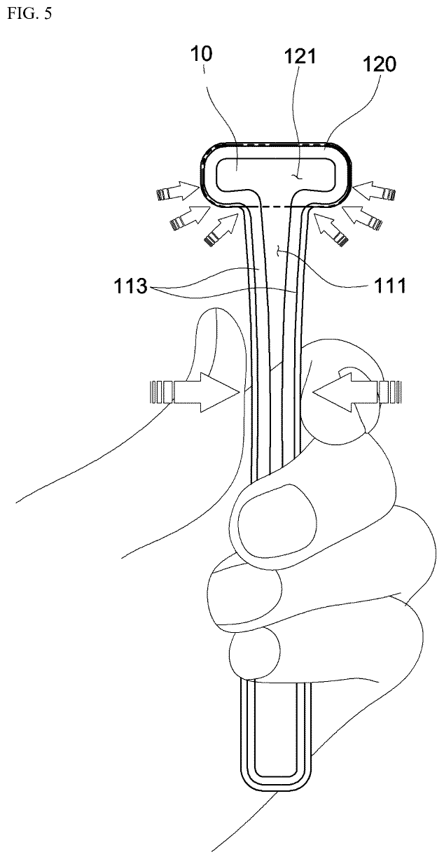

It is preferable that the main body (100) in the form of such a frame is made of a synthetic resin material that is hard but elastically deformable. Accordingly, as shown in FIG. 5, when the handle frame (110) is held by hand and the pair of vertical frames (113) are compressed inwardly, the head frame (120) is also configured to be compressively deformed in conjunction with the compressive deformation of the handle frame (110), and the head (10) coupled to the second mounting space (121) is compressed and fixed within the second mounting space (121).

This is because the first mounting space (111) and the second mounting space (121) are connected to each other to form a single channel, and also because the main body (100) itself is formed in a frame shape, and accordingly, due to the characteristics of the main body (100) that supports the head (10) with a structure that opens both the front and rear of the head (10), even if the coupling force with the head (10) is reduced when pressing against the tongue, the main body (100) naturally strongly presses and grips the edge of the head (10) in the process of sweeping the tongue using the tongue cleaner, so that the head (10) in the second mounting space (121) can be firmly fixed without being arbitrarily disassembled.

In addition, when attaching or detaching the head (10) coupled to the first mounting space (111) or the second mounting space (121), rather than relying solely on the elastic deformation of the head (10), the first mounting space (111) and the second mounting space (121) are expanded by moving the pair of vertical frames (113) outward, thereby making the attachment or detachment process of the head (10) easier.

In order to implement these structural features, it is preferable that the head (10) is made of a soft material, and the main body (100) has a frame shape made of a hard but elastically deformable material.

In addition, because the inside of the main body (100) is provided as an empty space, it not only provides ease of cleanliness care but also makes the tongue cleaner itself lighter and more simplified in structure, thereby ensuring profitability by drastically reducing production costs and improving convenience of use.

Meanwhile, a support (115) may be formed at the bottom of the first mounting space (111).

The support (115) is a component for reinforcing the strength of the lower part of the handle frame (110), and plays a role in stabilizing the structure of the main body (100) by limiting excessive elastic deformation of the vertical frame (113) during use of the tongue cleaner.

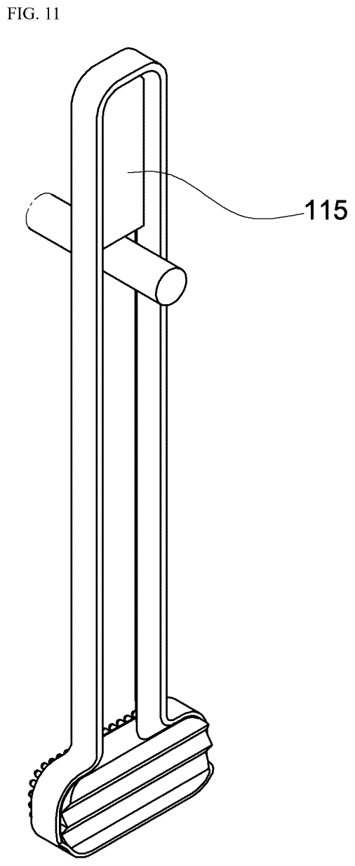

As shown in FIG. 11, the tongue cleaner may be stored by turning it over and hanging it on a hook or the like using the support (115).

Next, the head (10) may be detachably coupled to the mounting space and may have the form of a brush and scraper that cleans and massages the tongue.

In this case, a first protrusion (12) is formed on the front side of the head (10), and a second protrusion (13) is formed on the back of the head (10) in the front and rear directions, respectively, so that at least one of the first protrusion (12) and the second protrusion (13) can be used by contacting the tongue.

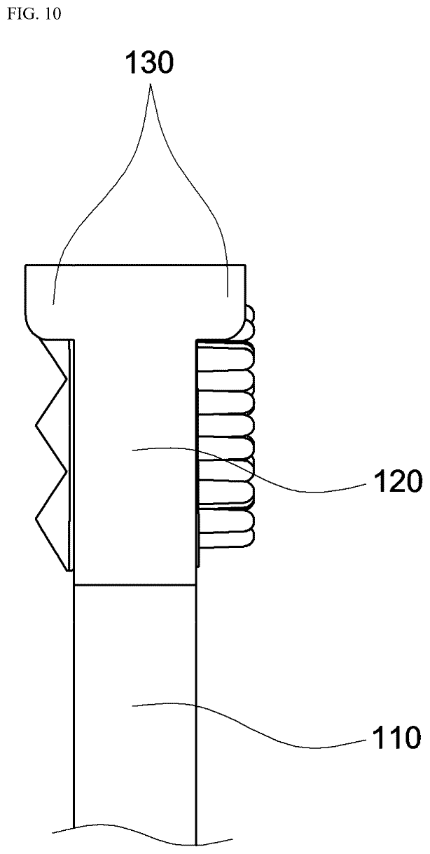

Meanwhile, in the basic embodiment, the scraper protrusion (130) is formed at the upper front side of the head frame (120), the action of the scraper protrusion (130) is excluded when performing hygiene care of the tongue with the rear configuration of the tongue cleaner, only the head (10) can be applied to the tongue. However, if necessary, as shown in FIG. 10, the scraper protrusions (130) can be formed symmetrically on the front and rear upper portions of the head frame (120) so that both sides of the tongue cleaner can be used for removing tongue plaque.

It is preferable that the first protrusion (12) and the second protrusion (13) are formed of protrusions of different shapes, and the shape or arrangement of the protrusions is not specifically limited, but the first protrusion (12) is formed as a protrusion of a triangular structure that is long from left to right so as to perform a function similar to that of the scraper protrusion (130), so that a plurality of protrusions can be formed at equal intervals from top to bottom.

In the case of the second protrusion (13), it is configured with a thin and long rod-shaped brush structure so that the soft tissue can be gently massaged and foreign substances between the fine lingual papillae of the tongue with minimal stimulation, and a plurality of them can be densely arranged.

In some cases, as shown in FIG. 10, a plate-shaped third protrusion (13a) can be mixed between the second protrusions (13) on the rear of the head (10) to ensure cleaning and massage efficiency by providing more complex stimulation.

The embodiments of the first protrusion (12) and second protrusion (13) as described above are merely exemplary, and it is revealed that the head (10) adopting various protrusion structures may be used.

The embodiments of the present disclosure described above are merely exemplary, and those skilled in the art to which the present disclosure pertains will appreciate that various modifications and other equivalent embodiments are possible.

Therefore, it will be well understood that the present disclosure is not limited to the forms mentioned in the detailed description above.

Therefore, the true technical protection scope of the present disclosure should be determined by the technical idea of the appended patent claims.

It is also to be understood that the present disclosure includes all modifications, equivalents, and alternatives falling within the spirit and scope of the present disclosure as defined by the appended claims.

### Description of the Sign

100: main body
10: head
110: handle Frame
120: head frame
130: scraper protrusion

1. A tongue cleaner comprising:
a hard body formed in a frame shape with a mounting space formed through the inside; and
a soft head that is detachably attached to said mounting space and perform tongue cleaning and massage;
wherein said hard body comprises:
a handle frame having a first mounting space formed inside; and a head frame formed extending from an upper end of said handle frame and having a second mounting space formed internally and communicating with the first mounting space; wherein said hard body having the frame shape is formed of an elastically deformable material, and said head frame is compressively deformed in conjunction with the handle frame according to the compressive deformation thereof, and the head coupled to the second mounting space is compressed and fixed within the second mounting space.

2. The tongue cleaner according to claim 1, characterized in that a scraper protrusion for removing a tongue plaque is formed on the upper end of said hard body.

3. The tongue cleaner according to claim 1, characterized in that at least one head is combined and stored in a row in said first mounting space.

4. A tongue cleaner characterized in that a support is formed at the lower portion of said first mounting space.
